(19) United States
(12) Patent Application Publication
(10) Pub. No.: US 2025/0387182 A1
(43) Pub. Date: Dec. 25, 2025

(54) ADJUSTABLE CAMERA REFERENCE SYSTEM AND METHOD FOR USE THEREOF

(71) Applicant: EMPLASE Medical Technologies, LLC, Cordova, TN (US)

(72) Inventors: Richard A. Hynes, Melbourne, FL (US); Matthew M. Morrison, Cordova, TN (US)

(21) Appl. No.: 19/305,704

(22) Filed: Aug. 20, 2025

Related U.S. Application Data
(63) Continuation of application No. 19/233,913, filed on Jun. 10, 2025.
(60) Provisional application No. 63/658,690, filed on Jun. 11, 2024.

(51) Int. Cl. A61B 34/30; A61B 34/00; A61B 34/20

(52) U.S. Cl. CPC A61B 34/30; A61B 34/20; A61B 34/25; A61B 2034/2057; A61B 2034/258

(57) ABSTRACT

An adjustable camera reference system and method for use thereof for maintaining guidance of a navigated instrument and/or an a navigated end effector during performance of surgery is provided. The adjustable camera reference system can employ one or more main reference cameras and various floating reference sources, and if views of various tracking markers positioned in an operating room by the one or more main reference cameras are interrupted or lost, positions of the one or more main reference cameras, and data and images provided by one or more of the various floating reference sources can be hot-swapped or substituted for data and images of the various tracking markers provided by the one or more main reference cameras. One or more of the various tracking markers are attached relative to the navigated instrument and/or the navigated end effector, and by determining the locations thereof, the proper guidance of the navigated instrument and/or the navigated end effector can be maintained.

The present application is a continuation-in-part of U.S. Ser. No. 19/233,913, filed Jun. 10, 2025; which claims the benefit of U.S. Provisional Application No. 63/658,690, filed Jun. 11, 2024; which are hereby incorporated by reference in their entirety.

### FIELD

The present disclosure is directed to an adjustable camera reference system and method for use thereof for maintaining guidance of a navigated instrument and/or a navigated end effector relative to a surgical site(s) on a patient using a navigation guidance system, and via autonomous, manual, or robotic adjustment of the adjustable camera reference system relative to the surgical site(s), the navigated instrument, and the navigated end effector using feedback from a control loop controlling operation of the navigation guidance system and the adjustable camera reference system for doing so and in order to optimize surgical workflow.

### BACKGROUND

Conventional camera reference systems have been used in determining relative locations of a patient and equipment in an operating room during performance of a surgery. In doing so, the conventional camera reference systems can be used to facilitate guidance of a navigated instrument and/or an end effector of a robotic armature. To illustrate, the conventional camera reference systems can supply input in the form of data and images of the patient and the equipment to navigation systems, and the navigation systems under direction of a surgeon(s) can be used to plan the surgery and to guide the navigated instrument and/or the end effector based on the supplied input from the conventional camera reference system. Conventional tracking (or reference) markers have been used to facilitate sensing and locating by the conventional camera reference systems and the navigation systems. The conventional tracking markers can be attached to portions of the patient and the equipment in the operating room, and can be configured to reveal the locations thereof. The conventional tracking markers can be viewed by main camera(s) of the conventional camera reference systems, and the data and images generated by the conventional camera reference systems can be inputted to the navigation system to identify the locations of the portions the patient and the equipment to which they are attached. However, such an arrangement has significant limitations. The operating room can be a busy place with body parts of the surgeon(s), nurse(s), and/or surgical assistant(s), and the equipment held by the same moving to partially or totally block the view of the main camera(s) of the conventional camera reference systems. Furthermore, additional equipment in the operating room can also partially or totally block the main camera(s) of the conventional camera reference systems during use thereof. And the blocking of the main camera(s) can serve in disrupting corresponding viewing of the conventional tracking markers. Such disruptions can interfere with the efficient operation of the navigation systems, because the locations of the conventional tracking markers may be lost to the navigation systems.

Therefore, there is a need for a system to maintain viewing of tracking markers even when main camera(s) are partially or totally blocked. As discussed below, an improved adjustable camera reference system and method for use thereof according to the present disclosure can be used to maintain provisioning of data and images of tracking markers to a navigation system even if main camera(s) of the improved adjustable camera reference system are partially or totally blocked. The improved adjustable camera reference system and method for use thereof according to the present disclosure can supply the data and images to the navigation system in order to maintain guidance of a navigated instrument and/or a navigated end effector relative to a surgical site(s), and a control loop for controlling operation of the navigation guidance system and the adjustable camera system can be used in autonomous, manual, or robotic adjustment of the improved adjustable camera system for doing so and in order to optimize surgical workflow.

### SUMMARY

This disclosure generally relates to an adjustable camera reference system and method of use thereof.

In one aspect, the present disclosure contemplates a method of performing navigated surgery, the method including positioning a patient on a surgical table or frame; attaching a first tracking marker to the patient adjacent to a surgical site on the patient; attaching a second tracking marker to one of a navigated instrument and a robotic-navigated end effector; attaching a third tracking marker in a fixed position relative to the surgical table or frame; sensing and identifying the first tracking marker, the second tracking marker, the third tracking marker, and relative locations thereof with at least one main reference camera or sensor; determining if the sensing and identifying of the first tracking marker, the second tracking marker, or the third tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker, the second tracking marker, and the third tracking marker; manipulating the position of the at least one main reference camera or sensor according to the adjustments and/or the articulations displayed by the control computer and/or the camera scale set-points controller; restoring the sensing and identifying of the first tracking marker, the second tracking marker, and the third tracking marker after the manipulation of the at least one main reference camera or sensor; generating and displaying computer-generated data and images from information provided by the at least one main reference camera or sensor including locations of the first tracking marker, the second tracking marker, and the third tracking marker relative to one another, to the patient, and to an ideal surgical approach; and adjusting the location of the one of the navigated instrument and the robotic-navigated end effector after comparing the location thereof relative to the ideal surgical approach.

In another aspect, the present disclosure contemplates a method of performing navigated surgery, the method including positioning a patient on a surgical table or frame; attaching a first tracking marker to the patient adjacent to a surgical site on the patient; attaching a second tracking marker to one of a navigated instrument and a robotic-navigated end effector; sensing and identifying the first tracking marker and the second tracking marker, and relative locations thereof with at least one main reference camera or sensor; determining if the sensing and identifying of the first tracking marker or the second tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker or the second tracking marker; manipulating the position of the at least one main reference camera or sensor according to the adjustments and/or the articulations displayed by the control computer and/or the camera scale set-points controller by at least one of lengthening or shortening a length of a post portion of the at least one main reference camera or sensor, rotating and/or tilting of at least one armature of the at least one main reference camera or sensor, and rotating and/or tilting of a camera portion of the at least one main reference camera or sensor; restoring the sensing and identifying of the first tracking marker or the second tracking marker after the manipulation of the at least one main reference camera or sensor; generating and displaying computer-generated data and images from information provided by the at least one main reference camera or sensor including locations of the first tracking marker and the second tracking marker relative to one another, to the patient, and to an ideal surgical approach; and adjusting the location of the one of the navigated instrument and the robotic-navigated end effector after comparing the location thereof relative to the ideal surgical approach.

In yet another aspect, the present disclosure contemplates a method of performing navigated surgery, the method including positioning a patient on a surgical table or frame; attaching a first tracking marker to the patient adjacent to a surgical site on the patient; attaching a second tracking marker to one of a navigated instrument and a robotic-navigated end effector; sensing and identifying the first tracking marker and the second tracking marker, and relative locations thereof with at least one main reference camera or sensor; determining if the sensing and identifying of the first tracking marker or the second tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker or the second tracking marker; manipulating the position of the at least one main reference camera or sensor according to the adjustments and/or the articulations displayed by the control computer and/or the camera scale set-points controller by at least one of lengthening or shortening a length of a post portion of the at least one main reference camera or sensor, rotating and/or tilting of at least one armature of the at least one main reference camera or sensor, and rotating and/or tilting of a camera portion of the at least one main reference camera or sensor; restoring the sensing and identifying of the first tracking marker or the second tracking marker after the manipulation of the at least one main reference camera or sensor; sensing and identifying the first tracking marker or the second tracking marker with at least one floating reference source attached adjacent one of the first tracking marker and the second tracking marker if the sensing and identifying of the first tracking marker or the second tracking marker by the at least one main reference camera or sensor is interrupted or lost; generating and displaying computer-generated data and images from information provided by the at least one main reference camera or sensor and the at least one floating reference source including locations of the first tracking marker and the second tracking marker relative to one another, to the patient, and to an ideal surgical approach; and adjusting the location of the one of the navigated instrument and the robotic-navigated end effector after comparing the location thereof relative to the ideal surgical approach.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is an exemplary depiction of an operating room according to an embodiment of the present disclosure including an embodiment of an interoperative patient positioning and manipulation (IPPM) system, a navigation guidance system, a navigated instrument, a stand-mounted camera of a camera reference system, and an intraoperative imaging system;

FIG. 2 is an exemplary depiction of an operating room according to an embodiment of the present disclosure including an embodiment of the IPPM system, the navigation guidance system, a robotic armature, an end effector of the robotic armature, the stand-mounted camera of the camera reference system, and various tracking markers attached relative to such equipment;

FIG. 3 is an exemplary depiction of a portion of the operating room of FIG. 2 depicting a portion of the patient, the end effector of the robotic armature positioned relative to the portion of the patient, a portion of the stand-mounted camera, and the various tracking markers attached relative to the patient and such equipment.

FIG. 4 depicts a schematic depiction of potential placement of equipment in the operating rooms of FIGS. 1 and 2, and potential placement of the various tracking markers relative to the patient and such equipment depicted in FIGS. 1 and 2;

FIG. 5 depicts a flow-chart detailing an embodiment of a control loop that can be used to facilitate navigation of the navigated instrument and the navigated end effector;

FIG. 6 depicts a flow-chart detailing an embodiment of the control loop that can be used to facilitate navigation of the navigated instrument and the navigated end effector, and, if necessary, hot-swapping or substituting data and images from one or more floating reference markers;

FIG. 7 depicts a diagrammatic depiction of the various floating reference sources that can be placed relative to equipment in the operating rooms of FIGS. 1 and 2, and a control system that can be used to control operation thereof; and

FIG. 8 depicts an upper portion of a version of a stand-mounted camera, a surface-mounted camera, and/or other sensors, and depicts various length and angular adjustments and articulations afforded thereby.

### DETAILED DESCRIPTION

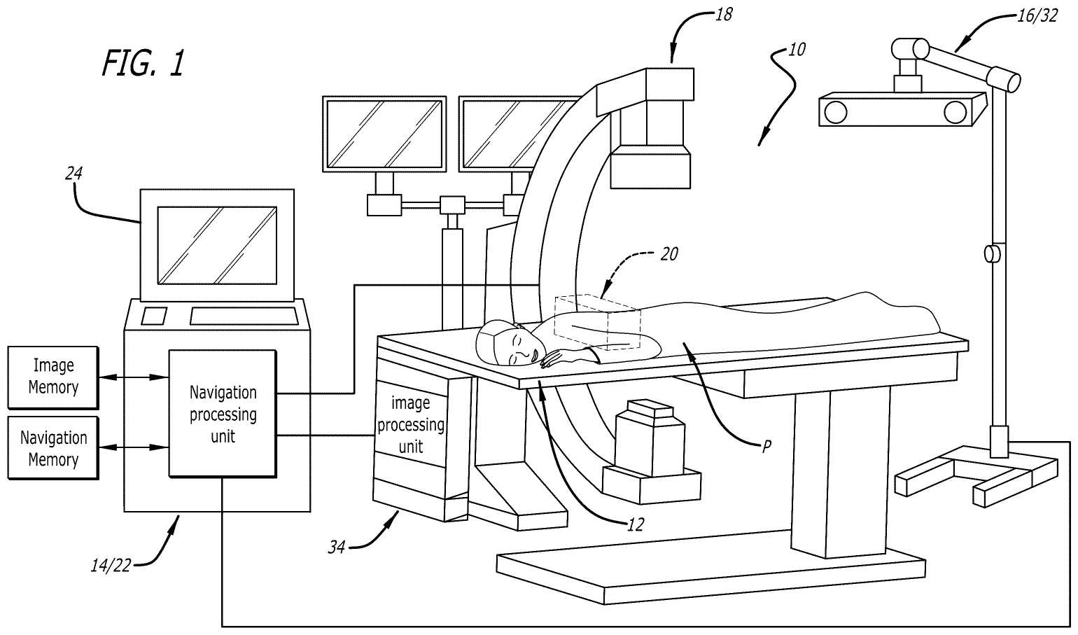

An operating room is generally indicated by the numeral 10 in FIG. 1, and the operating room 10 can include various equipment to facilitate performance of a surgical operation on a patient P. To illustrate, the operating room 10, as depicted in FIG. 1, can include a surgical table or a surgical frame (such as an interoperative patient positioning and manipulation (IPPM) system 12), a navigation system 14, a camera reference system 16, and an intraoperative imaging system 18.

The IPPM system 12 can be used to support and articulate the patient P during surgery. For example, the IPPM system 12 could be similar to patient-positioning systems disclosed in U.S. Pat. No. 10,966,892 and in U.S. Pat. Nos. 12,011,396, 12,011,397, and 12,011,398, which are hereby incorporated herein by reference. The IPPM system 12 can include a fixed or stationary portion and a moveable or repositionable portion, and the moveable or repositionable portion can be used to facilitate simultaneous surgical access to different surgical sites(s) on the patient P via movement thereof. Exemplary simultaneous surgical access is disclosed in U.S. Pat. No. 9,730,684, which is hereby incorporated by reference herein.

As depicted in FIG. 1, the navigation system 14 can include a navigated instrument 20, a control computer 22, at least one monitor 24, the camera reference system 16, and the intraoperative imaging system 18. As depicted in FIG. 1, the navigated instrument 20 is generally represented by a box adjacent surgical site(s) on the patient P, and the navigated instrument 20 can be many alternative instruments that can be manipulated by a surgeon (or surgeons) S during surgery relative to the surgical site(s). As discussed below, in other embodiments of the operating room 10, a surgical robot with an end effector can be provided in addition to or instead of the navigated instrument 20 manipulated by the surgeon S.

The control computer 22 can be used to control operation of navigation system 14, and in doing so, accept inputs from the camera reference system 16 and the intraoperative imaging system 18. Using the navigation system 14 (and the control computer 22), the navigated instrument 20 can be manipulated by the surgeon S under the guidance provided thereby to facilitate the surgery on the patient P. The at least one monitor 24 can display information regarding operation of the control computer 22 and/or data and images created by the navigation system 14 to facilitate such guidance. The inputs provided by the camera reference system 16 and the intraoperative imaging system 18 to the navigation system 14 allow, for example, positions of the navigated instrument 20 to be displayed on the at least one monitor 24 relative to data and images developed before or generated during surgery.

The camera reference system 16 can employ various passive or active tracking (or reference) markers 30 positioned in the operating room 10 to facilitate determination of relative positions thereof. As depicted in FIG. 1, the camera reference system 16 includes a stand-mounted camera 32 (FIGS. 1-5) that is moveable/adjustable relative the IPPM system 12 to facilitate positioning and repositioning thereof. The stand-mounted camera 32 can serve as the main reference camera or standard (MRS) for viewing portions of the patient P and the various tracking markers 30. Portions of the stand-mounted camera 32 can be manually or robotically controlled to position or reposition the camera thereof to provide line-of-sight with the various tracking markers 30. The stand-mounted camera 32 can include one or more visible-light, infrared cameras, and/or electromagnetic radiation sensors (such as, for example, multi-planar camera(s)) that can be used in generating data and images of the surgical site(s) and the various tracking markers 30, and for determining the relative positions of the various tracking markers 30. The data and images provided by the stand-mounted camera 32 can be used to facilitate generation of computer-generated 2D and/or 3D data (collectively “data”), and 2D and/or 3D images and/or video (collectively “images”) to facilitate performance of the surgery. Such computer-generated data and images can incorporate continuous real-time feeds from the one or more visible-light, infrared cameras, and/or electromagnetic radiation sensors. The computer-generated data and images, for example, can be used in building 2D and/or 3D maps that can include computer-generated indicia identifying the various tracking markers 30 provided over (or overlayed) the real-time feeds from the one or more visible-light, infrared cameras, and/or electromagnetic radiant sensors, and/or include entirely computer-generated graphics showing the same. The computer-generated data and images can be displayed on the at least one monitor 24 or other local/remote monitor(s).

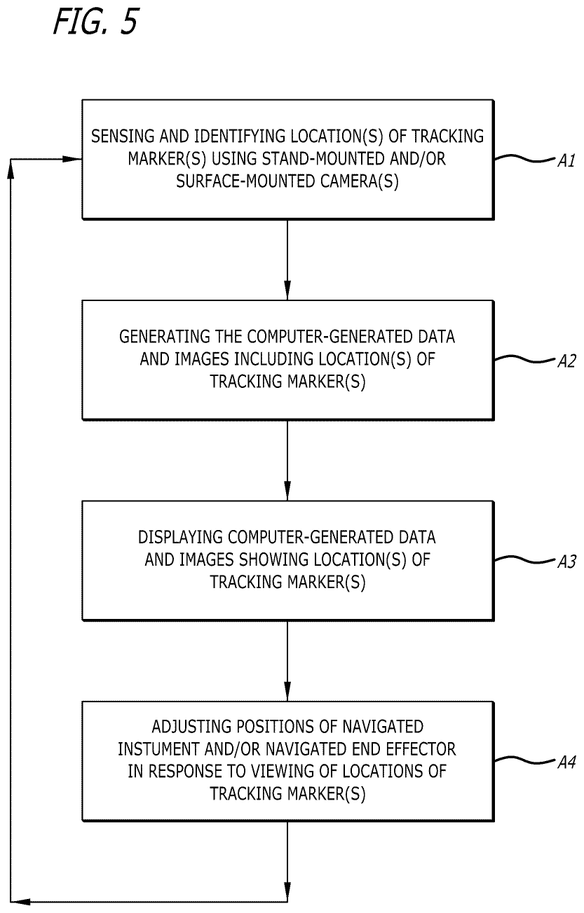

The feedback afforded by continuous generation of the data and images for determining the relative positions of the various tracking markers 30 using the stand-mounted camera 32 and the generated data and images of the surgical site(s) and the various tracking markers 30 facilitates creation of a control loop that affords proper positioning of the navigated instrument 20 relative to the surgical site(s). As depicted in FIG. 5, the control loop is afforded by (A₁) sensing and identifying the location of the various tracking markers 30 relative to one another and to the patient P using the stand-mounted camera 32, (A₂) generating the computer-generated data and images including the representations of the locations of the various tracking markers 30 relative to the patient P, (A₃) displaying the computer-generated data and images on the at least one monitor 24 or the other local/remote monitor(s), (A₄) adjusting of the navigated instrument 20 after viewing of the locations of the various tracking markers 30 relative to the patient P and to one another, and then continuously repeating actions (A₁), (A₂), (A₃), and (A₄) to properly position the navigated instrument 20 to effectuate performance of the surgery. While depicted in FIG. 5 as being separate, the actions (A₁), (A₂), (A₃), and (A₄) can be combined with one another. Furthermore, when using the navigated instrument 20, the surgeon S would control the adjustment of the navigated instrument 20 in response to the displayed computer-generated data and images. As discussed below, an ideal surgical approach, pathway, or trajectory can be determined using the navigation system 14, and the adjustment of the navigated instrument 20 can be in comparison to the ideal surgical approach, pathway, or trajectory displayed with or as part of the computer-generated data and images on the at least one monitor 24 or the other local/remote monitor(s).

Furthermore, the intraoperative imaging system 18 can be supported by or incorporated with a moveable supported structure 34 to facilitate positioning and repositioning thereof. Portions of the moveable support structure 34 can be manually on robotically (FIG. 1) controlled to position and reposition the intraoperative imaging system 18 relative to the patient P to provide at least imaging with respect to the surgical site(s). The intraoperative imaging system 18 can employ, for example, imaging systems using fluoroscopy, CT, MRI, ultrasound, and PET to develop data and images of the surgical site(s). Like the data and images generated by the stand-mounted camera 32, the data and images generated by the intraoperative imaging system 18 can be used in the above-described feedback loop.

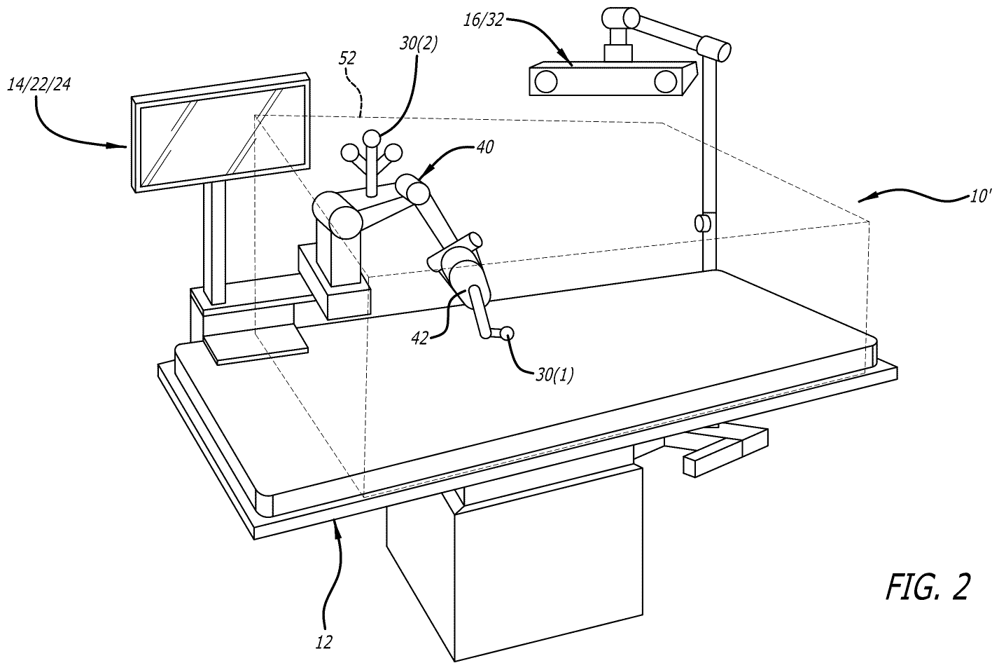

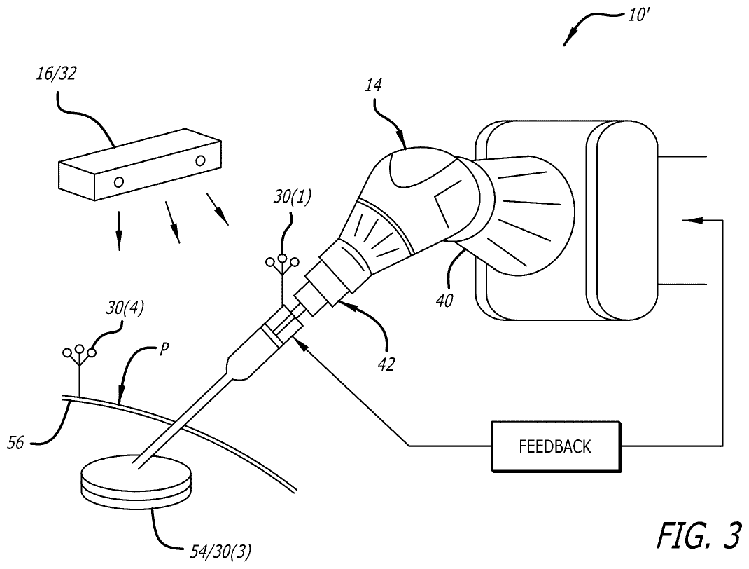

A modified operating room is generally indicated by the numeral 10′ in FIG. 2. Like the operating room 10, the modified operating room 10′ can include the IPPM system 12, the navigation system 14, and the stand-mounted camera 32. Although not shown in FIG. 2, the modified operating room 10′ also can include the intraoperative imaging system 18. In addition to or instead of the navigated instrument 20, the navigation system 14 can control and/or include a robotic armature 40. The robotic armature 40 can be supported by the IPPM system 12 and/or the navigation system 14. As depicted in FIG. 2, the robotic armature 40 is attached relative to the navigation system 14, and the navigation system 14 is attached relative to the IPPM system 12. The robotic armature 40, as depicted in FIGS. 2 and 3, can include a navigated end effector 42, and the navigated end effector 42 can include a surgical instrument that can be guided into position relative to the surgical site(s). Using the navigation system 14, the navigated end effector 42 can be positioned and repositioned relative to the surgical site(s) via actuation of the robotic armature 40. The navigated end effector 42 can be actuated into proper position relative to the surgical site(s) by the navigation system 14 using the control loop employing the above-discussed feedback.

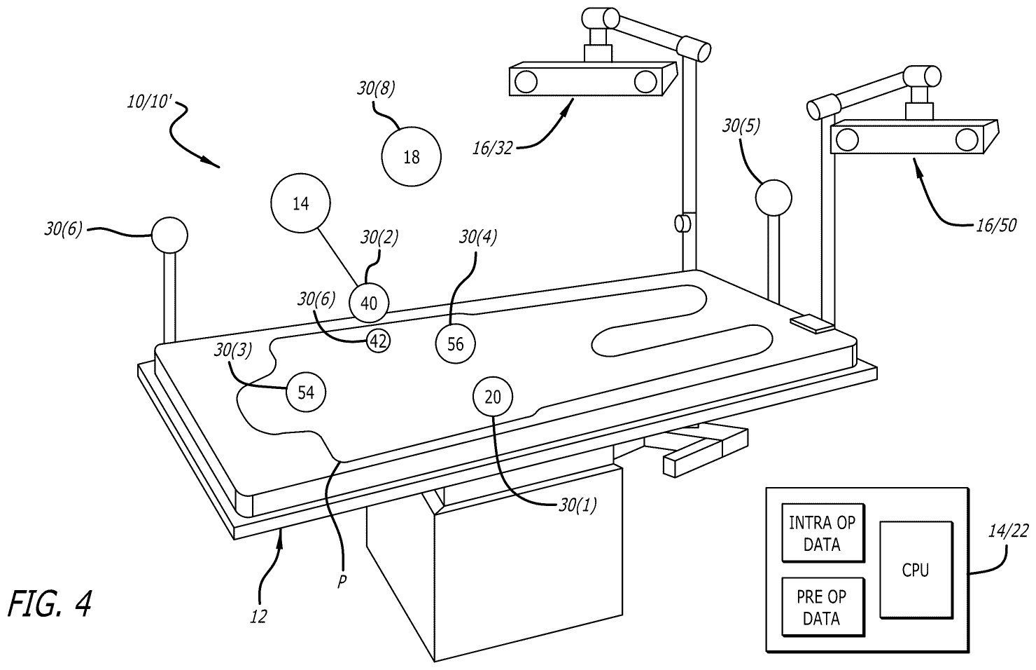

Whether using the operating room 10 or the modified operating room 10′, the camera reference system 16 can include a surface-mounted camera 50 in addition to or instead of the stand-mounted camera 32. The surface-mounted camera 50 (FIGS. 4 and 5) can be supported in the operating room 10 or the modified operating room 10′ via attachment to horizontal or vertical surfaces (such as a ceiling, a floor, or wall(s)) thereof, and/or attached to the IPPM system 12 and/or the navigation system 14 and can be used in similar fashion to the stand-mounted camera 32. The surface-mounted camera 50 can also serve as the main reference camera or standard (MRS) for viewing portions of the patient P and the various tracking markers 30. Like the stand-mounted camera 32, portions of the surface-mounted camera 50 can be manually or robotically adjusted to position or reposition the camera thereof to provide line-of-sight with the various tracking markers 30. Like the stand-mounted camera 32, the surface-mounted camera 50 can include one or more visible-light, infrared cameras, and/or electromagnetic radiation sensors (such as, for example, multi-planar camera(s)) that can be used in generating data and images of the surgical site(s) and the various tracking markers 30, and for determining the relative positions of the various tracking markers 30. The data and images provided by the surface-mounted camera 32 can be used to facilitate generation of computer-generated 2D and/or 3D data (collectively “data”), and 2D and/or 3D images and/or video (collectively “images”) to facilitate performance of the surgery. These computer-generated data and images can incorporate continuous real-time feeds from the one or more visible-light, infrared cameras, and/or electromagnetic radiation sensors. The computer-generated data and images, for example, can be used in building 2D and/or 3D maps that include computer-generated indicia identifying the various tracking markers 30 provided over (or overlayed) the real-time feeds from the one or more visible-light, infrared cameras, and/or electromagnetic radiant sensors, and/or include entirely computer-generated graphics showing the same. The computer-generated data and images can be displayed on the at least one monitor 24 or other local/remote monitor(s).

Like use of the stand-mounted camera 32, the feedback afforded by the continuous generation of the data and images for determining the relative positions of the various tracking markers 30 using the surface-mounted camera 50 and the generated data and images of the surgical site(s) and the various tracking markers 30 facilitates creation of the control loop that affords the above-described proper positioning of the navigated instrument 20 and/or the navigated end effector 42 relative to the surgical site(s). As depicted in FIG. 5, the control loop is afforded by (A₁) sensing and identifying the location of the various tracking markers 30 relative to the patient P using the surface-mounted camera 50, (A₂) generating the computer-generated data and images including the representations of the locations of the various tracking markers 30 relative to the patient P, (A₃) displaying the computer-generated data and images on the at least one monitor 24 or the other local/remote monitor(s), (A₄) adjusting the position of the navigated end effector 42 after viewing of the locations of the various tracking markers 30 relative to the patient P and to one another, and then continuously repeating actions (A₁), (A₂), (A₃), and (A₄) to properly position the navigated end effector 42 to effectuate performance of the surgery. While depicted in FIG. 5 as being separate, the actions (A₁), (A₂), (A₃), and (A₄) can be combined with one another. Furthermore, if both the stand-mounted camera 32 and the surface-mounted camera 50 are used, then the data and images provided by both can be dynamically synchronized and integrated into the feedback loop for more accuracy in determining the locations of the various tracking markers.

When using the navigated end effector 42, the navigation system 14 (and the control computer 22) with input from the surgeon S would control the adjustment of the navigated end effector 42. As discussed below, the ideal surgical approach, pathway, or trajectory can be determined using the navigation system 14, and the adjustment of the navigated end effector 42 can be in comparison to the ideal surgical approach, pathway, or trajectory displayed with or as part of the computer-generated data and images on the at least one monitor 24 or the other local/remote monitor(s). Moreover, if the navigated system 14 (and the control computer 22) relies on artificial intelligence for controlling movement of the navigated end effector 42, then action (3) can be modified to remove the need to display the computer-generated data and images, and action (4) can be modified to remove the need to view the computer-generated data and images. Instead, the navigation system 14 (and the control computer 20) can evaluate the computer-generated data and images, and correspondingly adjust the navigated end effector 42 without the need for continuously displaying and viewing.

Thus, whether using the stand-mounted camera 32, the surface-mounted camera 50, and/or a combination thereof, data and images generated of the surgical sites(s) and the various tracking markers 30 afford determinations regarding the relative positions of the various tracking markers 30, and the navigation system 14 can correspondingly generate 2D and/or 3D maps detailing the location of the navigated instrument 20 and/or the navigated end effector 42 relative to the surgical site(s) for display on the at least one monitor 24 and/or other local/remote monitor(s) to aid and/or control navigation of the navigated instrument 20 and/or the navigated end effector 42 during the performance of the surgery.

The navigation system 14 (and the control computer 22) can also process data and images of the patient developed before surgery to aid in planning of or for comparison during the surgery. In doing so, the surgical planning can be performed by the navigation system 14 (and the control computer 22) and/or other local/remote computer using data and images developed by imaging systems prior to surgery. Such imaging systems can include fluoroscopy, CT, MRI, ultrasound, and PET, and can be performed in the time period leading up to surgery, and even include use of the camera reference system 16 and/or the intraoperative imaging system 18 in the operating room 10 or the modified operating room 10′ immediately prior to surgery. The data and images developed prior to surgery can be used by the navigation system 14 (and the control computer 22) and/or the other local/remote computer to generate 2D and/or 3D maps of anatomical structures of the patient P. The computer-generated 2D and/or 3D maps can then be used by the navigation system 14 (and the control computer 22) and/or the other local/remote computer with input from the surgeon to develop a surgical plan with the ideal surgical approach, pathway, or trajectory to the surgical site, and in doing so, determine the ideal surgical approach, pathway, or trajectory of the navigated instrument 20 and/or the navigated end effector 42 given the anatomical structures of the patient P. To illustrate, the data and images developed prior to surgery can be used to generate 2D and/or 3D maps of the anatomy of the patient P including both interior and exterior anatomical structures thereof to develop the surgical plan.

Furthermore, data and images generated by the camera reference system 16 and the intraoperative imaging system 18 in the operating room 10 and/or the operating room 10′ can supplement the data and images developed prior to surgery to provide (A) current 2D and/or 3D maps of both of the interior and exterior anatomical structures of the patient P, and/or (B) current 2D and/or 3D maps of the locations of the various tracking markers 30 immediately prior to surgery to further aid in surgical planning. The computer-generated 2D and/or 3D maps generated using the above-discussed data and images can be displayed by the navigation system 14 (and the control computer 22) on the at least one monitor 24 or the other local/remote monitor(s) to facilitate updates to the surgical plan.

As discussed above, during performance of the surgery, the computer-generated 2D and/or 3D maps can be displayed to show real-time information to facilitate proper positioning of the navigated instrument 20 and/or the navigated end effector 42 relative to the surgical site(s). That is, continuous real-time updates to the 2D and/or 3D maps can be provided via use of the camera reference system 16 and the intraoperative imaging system 18 to locate the various tracking markers 30 (including those provided on the navigated instrument 20, on the navigated end effector 42, and at the surgical site(s)) during the performance of the surgery, and the data and images can be dynamically integrated with each other, and with the other data and images generated prior to the performance of the surgery. And these continuous real-time updates, along with the other data and images developed prior to the surgery, can be inputs to the control loop that affords the above-described proper positioning of the navigated instrument 20 and/or the navigated end effector 42 relative to the surgical site(s) during the performance of the surgery.

As discussed above, the camera reference system 16 (including the stand-mounted camera 32 and/or the surface-mounted camera 50) can be used to determine the relative positions with the various tracking markers 30. The stand-mounted camera 32 and/or the surface-mounted camera 50 of the camera reference system 16, as discussed above, serves as the MRS for determining the relative positions of the various tracking markers 30, and in doing so, the camera reference system 16 is calibrated to define an operating-space control volume 52 (FIG. 2) in which these relative locations can be identified thereby. As such, portions of the camera-reference system 16 and/or the IPPM system 12 can be positioned relative to another so that portions of the IPPM system 12 and the patient P supported by the IPPM system 12 are within the operating-space control volume 52. Furthermore, the navigated instrument 20 (via manipulation by the surgeon) and the navigated end effector 42 (via actuation of the robotic armature 40) can be moved into and out of the operating-space control volume 52. The data and images provided by camera reference system 16 and the intraoperative imaging system 18 can be used separately and/or can be dynamically integrated with each other, and with the other data and images generated prior to the performance of the surgery to depict the locations of the various tracking markers 30 with respect to the surgical sites(s) on the patient.

As discussed above, the various tracking markers 30 can require line-of-sight to be identified by the camera reference system 16 (via the stand-mounted camera 32 and/or the surface-mounted camera 50). When passive, the various tracking markers 30 can include reflective markers that reflect visible and/or infrared light, and when active, the various tracking markers 30 can include light-emitting markers emitting visible, infrared light, and/or other electromagnetic radiation. The identification of the various tracking markers 30 within the operating-space control volume 52 can be robust as long as line-of-sight is maintained with the camera reference system 16.

A first tracking marker 30(1) FIG. 2) or an array of first tracking markers 30(1) (FIG. 3) can be attached to the navigated instrument 20 and/or the navigated end effector 42; second tracking marker(s) (a single one or an array thereof) 30(2) (FIG. 2) can be attached to the navigation system 14 and/or the robotic armature 40, and third tracking marker(s) (a single one or an array thereof) 30(3) can be attached to the patient P at an anatomical target 54 (FIG. 3) adjacent the surgical site(s).

As depicted in FIG. 4, additional tracking markers 30 (a single one or an array thereof) can be attached relative to the patient P and the equipment in the operating room 10 and/or the operating room 10′. Locations of the equipment in the operating room 10 and/or the operating room 10′ are schematically depicted by circular representations in FIG. 4. For example, fourth tracking marker(s) 30(4) can be attached to one or more additional anatomical references 56 of the patient P; fifth tracking marker(s) 30(5) can be attached to the fixed or stationary portion of the IPPM system 12 to serve as a fixed table reference; sixth tracking marker(s) 30(6) can be attached the moveable or repositionable portion of the IPPM system 12 to serve as a repositionable table reference; seventh tracking marker(s) 30(7) can be attached to a room reference 58 in the operating room 10 or the modified operating room 10′ on, for example, the ceiling, the floor, or the wall(s) thereof; and eighth tracking marker(s) 30(8) can be attached to the intraoperative imaging system 18.

So long as the various tracking markers 30 remain in the operating-space control volume 52 and the stand-mounted camera 32 and/or the surface-mounted camera 52 are not partially or totally blocked), the tracking markers 30 can be identified by the camera-reference system 16 (using the stand-mounted camera 32 and/or the surface-mounted camera 50) and/or the intraoperative imaging system 18 to generate data and images for determining the relative positions of thereof, and by extension the relative positions of portions of the patient P and the equipment to which the tracking markers 30 are attached. As discussed above, the continuous generation of the data and images for determining the relative positions of the tracking markers 30 using the stand-mounted camera 32 and/or the surface-mounted camera 50 can afford proper positioning of the navigated instrument 20 and/or the navigated end effector 42 relative to the surgical site(s).

As described above, the IPPM system 12 can include the moveable or repositionable portion that can be used to facilitate simultaneous surgical access to different surgical sites(s) on the patient P. The moveable or repositionable portion of the IPPM system 12 can be used to position and reposition the patient P before, during, and after surgery. To illustrate, the moveable or repositionable portion of the IPPM system 12 can be rotated relative to the fixed or stationary portion to move the patient P between prone, lateral, and supine positions, and positions therebetween. The moveable or repositionable portion of the IPPM system 12 can also be used to articulate portions of the patient P. The rotation and/or the articulation of the patient P can be used to facilitate simultaneous access to both a first surgical site and a second surgical site on the patient P.

During the performance of the surgery, views of the various tracking markers 30 by the MRS (the stand-mounted camera 30 and/or the surface-mounted camera 50 of the camera reference system 16) can be partially or totally blocked by, for example, the surgeon S, nurse(s), and/or surgical assistant(s), and/or the equipment in the operating room 10 or the modified operating room 10′. Furthermore, during the rotation and/or the articulation of the patient P, some of the various tracking markers 30 such as those attached relative to the patient P (e.g., the third tracking markers 30(3) or the fourth tracking marks 30(4)) and those attached relative to the equipment (e.g., the first tracking marker(s) 30(1), the second tracking marker(s) 30(2), or the sixth tracking marker(s) 30(6)) can be moved outside the envelope of the operating-space control volume 52.

Typically, the operating-space control volume 52 defined by the camera reference system 16 will have constrained movement relative to the IPPM system 12 and/or the patient P. For example, the stand-mounted camera 32 typically can be moved/adjusted relative to the IPPM system 12 and the patient P, but such movement/adjustment would be constrained by obstacles in the operating room 10 or the modified operating room 10′. Furthermore, the surface-mounted camera 50 typically can be adjusted relative to the IPPM system 12 and the patient P, but such adjustment would be constrained by the attachment thereof to the ceiling, the floor, or the wall(s) of the operating room 10 or the modified operating room 10′. The movement/adjustment of the stand-mounted camera 32 can be manual or automated, and if automated, can be controlled using a robotic camera controller 60 or a camera scale set-points controller 62 (FIG. 4) that can be separate or incorporated in/with the control computer 22 of the navigation system 14. And the adjustment of the surface-mounted camera 50 also can be manual or automated, and if automated, can be controlled using the robotic camera controller 60 ort the camera scale set-points controller 62. Such movement and/or adjustment of the stand-mounted camera 32 and/or the surface-mounted camera 50 may still not restore viewing of the various tracking markers 30 because of a partial or complete blockage thereof, or the various tracking markers 30 remain outside the operating-space control volume 52.

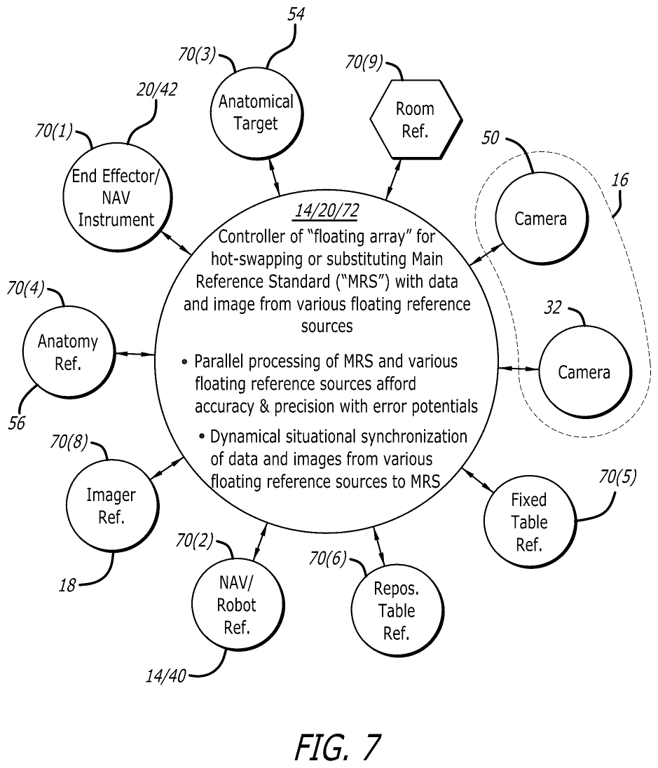

Line-of-sight to the various corresponding tracking markers 30 could be lost or interrupted until the stand-mounted camera 32 and/or the surface-mounted camera 50 are unblocked or those tracking markers 30 reenter the operating-space control volume 52. To illustrate, the rotation and/or the articulation of the patient P by the IPPM system 12, and articulation of the equipment (e.g., the robotic armature 40) could cause partial or total blockage of the various tracking markers 30 or movement of the tracking markers 30 outside the operating-space control volume 52 that cause loss or interruption of line-of-sight between the various corresponding tracking markers 30 and the camera reference system 16. Such loss or interruptions are obviously undesirable. As discussed below, an array of various floating reference sources 70, as depicted in FIG. 7, can be used by the camera reference system 16 to provide redundancy and compensate for the above-discussed loss or interruption to facilitate determination of the relative positions of the tracking markers 30 during such loss or interruption. As discussed below, if sensing of one of more of the tracking markers 30 by the camera reference system 16 (using the stand-mounted camera 32 or the surface-mounted camera 50) is lost or interrupted, the camera reference system 16 can hot-swap or substitute data and images provided by one or more of the various floating reference sources 70. The hot-swapping or the substitution affords “floating” or switching to the data and images provided by the one of more of various floating reference sources 70 best suited to view the one or more of the tracking markers 30 for which sensing is lost or interrupted. The best-suited of the various floating reference sources 70 could be a single one or an array of selected ones thereof.

The various floating reference sources 70 can be interconnected with a floating-reference control computer 72 to control operation thereof that communicates with the control computer 22 of the navigation system 14, or alternatively, the control computer 22 of the navigation system 14 can control operation of the floating reference sources 70 directly to afford such “floating” or switching. In addition to facilitating guidance of the navigated instrument 20 and the navigated end effector 42, the various floating reference sources 70 could also be used to guide manual or automated movement/adjustment of the stand-mounted camera 32 or the surface-mounted camera 50 to compensate for the above-discussed loss or interruption.

The various floating reference sources 70, for example, could be visible-light and/or infrared cameras (such as, for example, multi-planar camera(s)) that could be attached relative to the equipment or the patient P in the operating room 10 or the modified operating room 10′ and that can be used in generating the data and images of the surgical sites(s) and the various tracking markers 30. In addition to or instead of the visible-light and/or infrared cameras, the various floating reference sources 70 could also be electromagnetic radiation sensors that could be attached relative to the equipment or the patient P in the operating room 10 or the modified operating room 10′ and that can be used in generating the data and images of the surgical site(s) and the various tracking markers 30. Either way, the various floating reference sources 70 can be used to detect the location of the various tracking markers 30 whether the markers 30 are passive or active. Furthermore, as depicted in FIG. 7, the various floating reference sources 70 could be attached to or adjacent the navigated instrument 20 and/or the navigated end effector 42 (source 70(1)), the navigation system 14 and/or the robotic armature 40 thereof (source 70(2)), the anatomical target 54 (source 70(3)), the additional anatomical references 56 (source 70(4)), the fixed or stationary portion of the IPPM system 12 (source 70(5)), the moveable or repositionable portion of the IPPM system 12 (source 70(6)), the room reference 58 (source 70(7)), and/or the intraoperative imaging system 18 (source 70(8)). Additionally, a source 70(9) can be attached relative to the ceiling, the floor, or the wall(s) of the operating room 10 or the modified operating room 10″ to provide a source covering all or signification portions thereof,

The various floating reference sources 70 can generate the data and images for inputting into the floating-reference control computer 72, and the floating-reference control computer 72 can communicate these inputs to the control computer 22. Or, in addition to or instead of inputting to the floating-reference control computer 72, the data and images from the various floating reference sources 70, like the inputs from the camera reference system 16 (the stand-mounted camera 32 and/or the surface-mounted camera 50), can be inputted into the navigation system 14 (and the control computer 22). Preferably, these inputs can be sent and received wirelessly via wireless connections between the various floating reference sources 70, the floating-reference control computer 72, and/or the navigation system 14 (and the control computer 22). As discussed below, during performance of the surgery, the data and images from the various floating reference sources 70 can be incorporated into the above-discussed computer-generated 2D and/or 3D maps to locate the various tracking markers 30.

The control computer 22 can be configured to recognize if signal(s) are lost or interrupted between the camera reference system 16 and one or more of the various tracking markers 30, and then switch to one or more of the various floating reference sources 70 to supply data and images for determining the relative positions of the one or more tracking markers 30 for which signals to the camera reference system 16 are lost or interrupted to maintain tracking thereof. Such switching can be autonomous, and could occur automatically when the signals are lost or interrupted. And also upon recognition if signal(s) are lost or interrupted, the control computer 22 also can provide an indication that the stand-mounted camera 32 or the surface-mounted camera system 50 require manual or automated movement/adjustment and facilitate such movement/adjustment thereof to reposition the operating-space control volume 52 (via, for example, actuation initiated by the robotic camera controller 60 and the camera scale set-points controller 62.

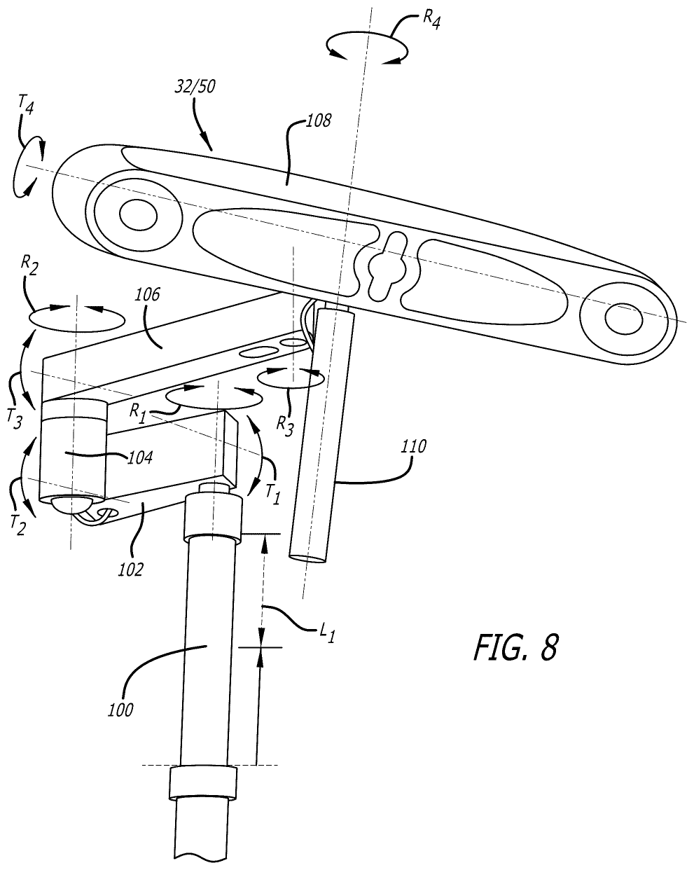

To illustrate, when the movement/adjustment is manually controlled, the control computer 22 and/or the camera scale set-points controller 62 can output various set-points for various possible length and angular adjustments and/or articulations of the stand-mounted camera 32 and the surface-mounted camera 50. FIG. 8 depicts an upper portion of a version of the stand-mounted camera 32, the surface-mounted camera 50, and/or other sensors, and depicts the various length and angular adjustments and articulations afforded thereby. The various length and angular adjustments and articulations of the stand-mounted camera 32, the surface-mounted camera 50, and/or other sensors allow positioning and repositioning thereof in tight spaces in the operating room 10 or the operating room 10′ to bring pertinent portions of the patient P, the surgical site(s), and/or surgical equipment (e.g., the navigated instrument 20 and/or the navigated end effector 42) into view. As depicted in FIG. 8, the stand-mounted camera 32, the surface-mounted camera 50, and/or other sensors can include a post portion 100, a first armature portion 102, a hinge portion 104, a second armature portion 106, a camera portion 108, and an adjustment handle 110.

The first armature portion 102 is supported by the post portion 100, the hinge portion 104 is attached to the first armature portion 102, the second armature portion 106 is supported by the hinge portion 104, and the camera portion 108 is supported by the second armature portion 106. As depicted in FIG. 8, the first armature portion 102 is rotatable and tiltable relative to the post portion 100, the second armature portion 106 is rotatable and tiltable relative to the hinge portion 104 (and the first armature portion 102), the camera portion 108 is rotatable and tiltable relative to the second armature portion 106, and the camera portion 108 is further rotatable and tiltable. More specifically, a length of the vertical portion 100 can be adjusted via lengthening or shortening thereof as indicated by a length adjustment L₁; a rotational angle of the first armature portion 102 relative to the post portion 100 can by adjusted as indicated by an rotational articulation R₁ and a tilt angle of the first armature portion 102 relative to the post portion 100 can be adjusted as indicated by a tilt articulation T₁; a tilt angle of the hinge portion 104 relative to the first armature portion 102 can be adjusted as indicated by a tilt articulation T₂; a rotational angle of the second armature portion 106 relative to the hinge portion 104 (and the first armature portion 102) can be adjusted as indicated by a rotational articulation R₂ and a tilt angle of the second armature portion 106 relative to the hinge portion 104 can be adjusted as indicated by a tilt articulation T₃; a rotational angle of the camera portion 108 relative to the second armature portion 106 can be adjusted as indicated by a rotational articulation R₃; and a rotational angle of the camera portion 108 can be further adjusted as indicated by a rotational articulation R₄ and a tilt angle of the camera portion 108 can be further adjusted as indicated by a tilt articulation T₄. The rotational articulations R₁, R₂, R₃, and R₄, and the tilt articulations T₁, T₂, T₃, and T₄ can occur about corresponding axes depicted in FIG. 8.

Although not shown in FIG. 8, scales for facilitating the length adjustment L₁, the rotational articulations R₁, R₂, R₃, and R₄, and/or the tilt articulations T₁, T₂, T₃, and T₄ can be provided on portions of the post portion 100, the first armature portion 102, the hinge portion 104, the second armature position 106, and the camera portion 108. The scales can correspond to and coordinate with the set-points outputted by the control computer 22 and/or the camera scale set-points controller 62 so that the user can make corresponding and coordinated adjustments and articulations of the length adjustment L₁, the rotational articulations R₁, R₂, R₃, and R₄, and/or the tilt articulations T₁, T₂, T₃, and T₄. More specifically, the set-points can specify a desired length adjustment L₁, desired rotational articulations R₁, R₂, R₃, and R₄, and/or a desired tilt articulations T₁, T₂, T₃, and T₄ for the post portion 100, the first armature portion 102, the hinge portion 104, the second armature portion 106, and the camera portion 108 of the stand-mounted camera 32, the surface-mounted camera 50, and/or other sensors. These set-points, for example, can be indicated and displayed to the user by the control computer 22 and/or the camera set-points controller 62 in measurements such as lengths and degrees that correspond to and coordinate with the scales, and the user can manually adjust and manipulate the above-discussed the camera portion 110 into position using the handle portion 110.

The above-discussed manual articulations adjustments can also be automated using various actuators (not shown) actuated by the user, the control computer 22, and/or the camera set-points controller 62 to control adjustments and articulations of the post portion 100, the first armature portion 102, the hinge portion 104, the second armature portion 106, and the camera portion 108 (e.g., the length adjustment L₁, the rotational articulations R₁, R₂, R₃, and R₄, and the tilt articulations T₁, T₂, T₃, and T₄). Additionally, the control computer 22 and/or the camera set-points controller 62 can also specify a location where the stand-mounted camera 32 should be moved in the operating room 10 or the modified operating room 10′. The operating-space control volume 52 can thereby be repositioned by such manual movement/adjustment according to the guidance provided by the control computer 22 and/or the camera set-points controller 62. Alternatively, the movement/adjustment of the stand-mounted camera 32 or the surface-mounted camera 50 to reposition the operating-space control volume 52 can occur automatically according to the indications from the control computer 22, the camera set-points controller 62, and/or the robotic camera controller 60 when such movement/adjustment is robotically controlled.

The control computer 22 can hot-swap or substitute the identification of one or more of the tracking markers 30 using the camera reference system 16 to the data and images for determining the relative positions of the one or more of the tracking markers 30 supplied by the various floating reference sources 70. The inputs to the control computer 22 from the camera reference system 16 and from the various floating reference sources 70 can be monitored and processed parallelly, so that the hot-swapping or substituting can occur instantaneously and the determination of the relative positions of the tracking members 30 can continue uninterrupted. In other words, the inputs from the camera reference system 16 and from the various floating reference sources 70 can be monitored in parallel to facilitate synchronization with one another, so that, after the tracking of one or more of the various tracking markers 30 by the camera reference system 16 is lost or interrupted, the tracking (via generation of the data and images) supplied by the various floating reference sources 70 can be exchanged therefor.

A temporary switch to one or more of the various floating reference sources 70 allows the location of the various tracking members 30 to be recalculated and remain known when the tracking of one or more of the various tracking markers 30 by the camera reference system 16 is lost or interrupted. The temporary switch afforded by the hot-swapping or substituting serves in decreasing error potentials during the determination of the relative positions of the tracking markers 30. To illustrate, even if the tracking of the tracking marker(s) 30(1) attached to the navigated instrument 20 and/or the navigated end effector, and/or the third tracking marker(s) 30(3) attached to the patient P at the anatomical target 54 adjacent the surgical site(s) is lost or interrupted, the navigation system 14 (and the control computer 22) can switch to one or more of the various floating reference sources 70 to track the tracking marker(s) 30(1) and 30(3) to maintain tracking thereof to ensure positional accuracy and precision of the position of the navigated instrument 20 and/or the navigated end effector 42 relative to the surgical site(s).

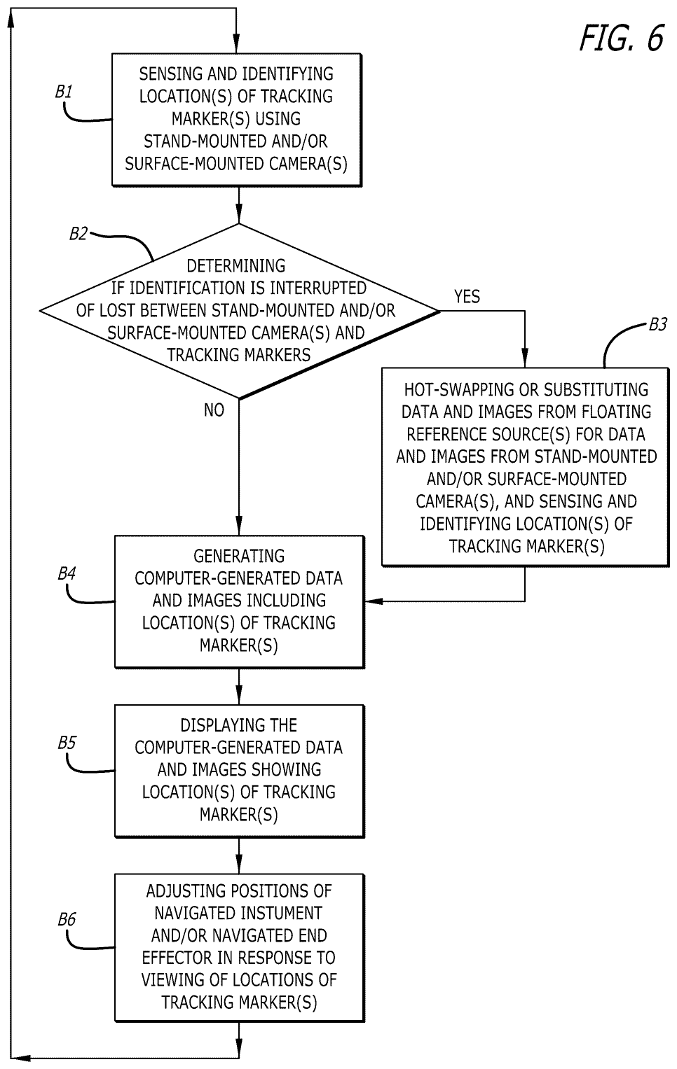

Like use of the stand-mounted camera 32 or the surface-mounted camera 50, the feedback afforded by the continuous generation of the data and images for determining the relative positions of the various tracking markers 30 using the various floating reference sources 70 creates a control loop (FIG. 6) that affords the above-described proper positioning of the navigated instrument 20 and/or the navigated end effector 42 relative to the surgical site(s) even when line-of-sight to the various corresponding tracking markers 30 is lost or interrupted to the camera reference system 16.

As depicted in FIG. 6, the control loop is afforded by (B₁) sensing and identifying of the location of the various tracking markers 30 relative to one another to the patient P using the stand-mounted camera 16, the surface-mounted camera 50, and/or the various floating reference sources 70, (B₂) determining if sensing and identification is lost between the stand-mounted camera 16 and/or the surface-mounted camera 50 and the various tracking markers 30, and if sensing and identification is lost or interrupted, then (B₃) hot-swapping or substituting the data and images from one or more of the various floating reference sources 70 for the corresponding data and images from the stand-mounted camera 16 and/or the surface-mounted camera, (B₄) generating the computer-generated data and images including the representations of the locations of the various tracking markers 30 relative to the patient P, (B₅) displaying the computer-generated data and images on the at least one monitor 24 or the other local/remote monitor(s), (B₆) adjusting the position of the navigated end effector 42 after viewing of the locations of the various tracking markers 30 relative to the patient P, and then continuously repeating actions (B₁), (B₂), (B₃), (B₄, (B₅), and (B₆) to properly position the navigated end effector 42 to effectuate performance of the surgery. While depicted in FIG. 6 as depicted in FIG. 6 as being separate, the actions (B₁), (B₂), (B₃), (B₄, (B₅), and (B₆) can be combined with one another.

The data and images from a single one or an array of the various floating reference markers 70 can be dynamically synchronized and integrated with the data and images from the stand-mounted camera 32 and/or the surface-mounted camera 50, and incorporated into the feedback loop for more accuracy in determining the locations of the various tracking markers 30. During performance of the surgery, the data and images from the various floating reference sources can be incorporated, as discussed above with respect to the data and images from the stand-mounted camera 32, the surface-mounted camera 50, into the above-discussed computer-generated 2D and/or 3D maps to locate the various tracking markers 30 for display on the at least one monitor 24 or other local/remote monitor(s). Furthermore, as discussed above, the ideal surgical approach, pathway, or trajectory can be determined using the navigation system 14, and the adjustment of the navigated instrument 20 or the navigated end effector 42 can be in comparison to the ideal surgical approach, pathway, or trajectory displayed with or as part of the computer-generated data and images on the at least one monitor 24 or the other local/remote monitor(s).

As discussed above, the data and images from the various floating reference sources 70 can be used to ensure positional accuracy and precision of the position of the navigated instrument 20 and/or the navigated end effector 42 relative to the surgical site(s). Such data and images from the various floating reference sources 70 and the control loop can also be used to manually or robotically guide movement/adjustment as described above of the stand-mounted camera 32 or the surface-mounted camera 50 to compensate for the above-discussed loss or interruption. Accordingly, the control loop of FIG. 6 improves robotic or navigation guidance to optimize surgical workflow by preventing interruption thereof.

It should be understood that various aspects disclosed herein may be combined in different combinations than the combinations specifically presented in the description and the accompanying drawings. It should also be understood that, depending on the example, certain acts or events of any of the processes of methods described herein may be performed in a different sequence, may be added, merged, or left out altogether (e.g., all described acts or events may not be necessary to carry out the techniques). In addition, while certain aspect of this disclosure are described as being performed by a single module or unit for purposes of clarity, it should be understood that the techniques of this disclosure may be performed by a combination of units or modules associated with the adjustable camera reference system and method for use thereof.

We claim:

1. A method of performing navigated surgery, the method comprising:
positioning a patient on a surgical table or frame;
attaching a first tracking marker to the patient adjacent to a surgical site on the patient;
attaching a second tracking marker to one of a navigated instrument and a robotic-navigated end effector;
attaching a third tracking marker in a fixed position relative to the surgical table or frame;
sensing and identifying the first tracking marker, the second tracking marker, the third tracking marker, and relative locations thereof with at least one main reference camera or sensor;
determining if the sensing and identifying of the first tracking marker, the second tracking marker, or the third tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker, the second tracking marker, and the third tracking marker;
manipulating the position of the at least one main reference camera or sensor according to the adjustments and/or the articulations displayed by the control computer and/or the camera scale set-points controller;
restoring the sensing and identifying of the first tracking marker, the second tracking marker, and the third tracking marker after the manipulation of the at least one main reference camera or sensor;
generating and displaying computer-generated data and images from information provided by the at least one main reference camera or sensor including locations of the first tracking marker, the second tracking marker, and the third tracking marker relative to one another, to the patient, and to an ideal surgical approach; and
adjusting the location of the one of the navigated instrument and the robotic-navigated end effector after comparing the location thereof relative to the ideal surgical approach.

2. The method of claim 1, wherein the at least one main reference camera or sensor includes a post portion, at least one armature portion interconnected with the post portion, and a camera portion interconnected with the armature portion, the post portion having an adjustable length, the at least one armature portion being rotatable and tiltable relative to the post portion, and the camera portion being rotatable and tiltable relative to the armature portion.

3. The method of claim 2, wherein the adjustments and/or the articulations displayed by the control computer and/or the camera scale set-points controller correspond to length adjustments to the post portion, first rotational and tilt adjustments to the at least one armature portion, and second rotational and tilt adjustments to the camera portion.

4. The method of claim 3, wherein the first tracking marker is attached adjacent an incision made in the patient to facilitate performance of the surgery therethrough, and the length adjustments, the first rotational and tilt adjustments, and the second rotational and tilt adjustments bring the first tracking marker, the second tracking marker, and the third tracking marker into view of the at least one main reference camera or sensor.

5. The method of claim 4, wherein each of the first tracking marker, the second tracking marker, and the third tracking marker are one of active and passive.

6. The method of claim 5, wherein, when passive, the first tracking marker, the second tracking marker, and the third tracking marker are reflective of visible light and/or infrared light, and when active, the first tracking marker, the second tracking marker, and the third reference marker emitting visible light, infrared light, and/or other electromagnetic radiation.

7. The method of claim 6, wherein the at least one main reference camera or sensor is one of a visible light camera, an infrared camera, and an electromagnetic sensor.

8. The method of claim 1, wherein the information provided by the at least one main reference camera or sensor includes data and images of the first tracking marker, the second tracking marker, the third tracking marker, and portions of the patient during the sensing and identifying thereof to facilitate the generating and displaying of the computer-generated data and images.

9. The method of claim 8, further comprising sensing and identifying the first tracking marker, the second tracking marker, or the third tracking marker with at least one floating reference source attached adjacent one of the first tracking marker, the second tracking marker, and the third tracking marker if the sensing and identifying of the first tracking marker, the second tracking marker, or the third tracking marker by the at least one main reference camera or sensor is interrupted or lost.

10. The method of claim 9, wherein the information provided by the at least one floating reference source includes data and images of at least one of the first tracking marker, the second tracking marker, and the third tracking marker, and the data and images provided by the at least one floating reference source is swapped or substituted for the data and images from the at least one main reference camera or sensor generated by the sensing and identifying of the first tracking marker, the second tracking marker, or the third tracking marker when the sensing and identifying is interrupted or lost.

11. The method of claim 10, wherein the at least one floating reference source includes a first floating reference source and a second floating reference source, and further comprising determining which of the first floating reference and the second floating reference have a better view of the first tracking marker, the second tracking marker, or the third tracking marker for which the sensing and identifying is interrupted or lost, and using the first floating reference and the second floating reference with the better view for the sensing and identifying.

12. A method of performing navigated surgery, the method comprising:
positioning a patient on a surgical table or frame;
attaching a first tracking marker to the patient adjacent to a surgical site on the patient;
attaching a second tracking marker to one of a navigated instrument and a robotic-navigated end effector;
sensing and identifying the first tracking marker and the second tracking marker, and relative locations thereof with at least one main reference camera or sensor;
determining if the sensing and identifying of the first tracking marker or the second tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker or the second tracking marker;
manipulating the position of the at least one main reference camera or sensor according to the adjustments and/or the articulations displayed by the control computer and/or the camera scale set-points controller by at least one of lengthening or shortening a length of a post portion of the at least one main reference camera or sensor, rotating and/or tilting of at least one armature of the at least one main reference camera or sensor, and rotating and/or tilting of a camera portion of the at least one main reference camera or sensor;
restoring the sensing and identifying of the first tracking marker or the second tracking marker after the manipulation of the at least one main reference camera or sensor;
generating and displaying computer-generated data and images from information provided by the at least one main reference camera or sensor including locations of the first tracking marker and the second tracking marker relative to one another, to the patient, and to an ideal surgical approach; and
adjusting the location of the one of the navigated instrument and the robotic-navigated end effector after comparing the location thereof relative to the ideal surgical approach.

13. The method of claim 12, wherein the first tracking marker is attached adjacent an incision made in the patient to facilitate performance of the surgery therethrough, and the at least one of lengthening or shortening the length of the post portion, the rotating and/or the tilting of the at least one armature, and the rotating and/or the tilting of the camera portion bring the first tracking marker and the second tracking marker into view of the at least one main reference camera or sensor.

14. The method of claim 13, wherein each of the first tracking marker, the second tracking marker, and the third tracking marker are one of active and passive.

15. The method of claim 14, wherein, when passive, the first tracking marker, the second tracking marker, and the third tracking marker are reflective of visible light and/or infrared light, and when active, the first tracking marker, the second tracking marker, and the third reference marker emitting visible light, infrared light, and/or other electromagnetic radiation.

16. The method of claim 15, wherein the at least one main reference camera or sensor is one of a visible light camera, an infrared camera, and an electromagnetic sensor.

17. The method of claim 16, further comprising sensing and identifying the first tracking marker or the second tracking marker with at least one floating reference source attached adjacent one of the first tracking marker and the second tracking marker if the sensing and identifying of the first tracking marker or the second tracking marker by the at least one main reference camera or sensor is interrupted or lost.

18. The method of claim 17, wherein the information provided by the at least one main reference camera or sensor and by the at least one floating reference source includes data and images of the first tracking marker, and the second tracking marker, and portions of the patient during the sensing and identifying thereof to facilitate the generating and displaying of the computer-generated data and images.

19. A method of performing navigated surgery, the method comprising:
positioning a patient on a surgical table or frame;
attaching a first tracking marker to the patient adjacent to a surgical site on the patient;
attaching a second tracking marker to one of a navigated instrument and a robotic-navigated end effector;
sensing and identifying the first tracking marker and the second tracking marker, and relative locations thereof with at least one main reference camera or sensor;
determining if the sensing and identifying of the first tracking marker or the second tracking marker by the at least one main reference camera or sensor is interrupted or lost, and if interrupted or lost, generating and displaying by a control computer and/or a camera scale set-points controller adjustments and/or articulations needed to manipulate the at least one main reference camera or sensor into position to restore the sensing and identifying of the first tracking marker or the second tracking marker;
manipulating the position of the at least one main reference camera or sensor according to the adjustments and/or the articulations displayed by the control computer and/or the camera scale set-points controller by at least one of lengthening or shortening a length of a post portion of the at least one main reference camera or sensor, rotating and/or tilting of at least one armature of the at least one main reference camera or sensor, and rotating and/or tilting of a camera portion of the at least one main reference camera or sensor;
restoring the sensing and identifying of the first tracking marker or the second tracking marker after the manipulation of the at least one main reference camera or sensor;
sensing and identifying the first tracking marker or the second tracking marker with at least one floating reference source attached adjacent one of the first tracking marker and the second tracking marker if the sensing and identifying of the first tracking marker or the second tracking marker by the at least one main reference camera or sensor is interrupted or lost;
generating and displaying computer-generated data and images from information provided by the at least one main reference camera or sensor and the at least one floating reference source including locations of the first tracking marker and the second tracking marker relative to one another, to the patient, and to an ideal surgical approach; and
adjusting the location of the one of the navigated instrument and the robotic-navigated end effector after comparing the location thereof relative to the ideal surgical approach.

20. The method of claim 19, wherein the first tracking marker is attached adjacent an incision made in the patient to facilitate performance of the surgery therethrough, and the at least one of lengthening or shortening the length of the post portion, the rotating and/or the tilting of the at least one armature, and the rotating and/or the tilting of the camera portion bring the first tracking marker and the second tracking marker into view of the at least one main reference camera or sensor.
